(19) United States
(12) Patent Application Publication
(10) Pub. No.: US 2026/0269652 A1
(43) Pub. Date: Sep. 10, 2026

(54) DIGITAL CONTROL AND ACTIVE TUNING FOR A WIRELESS CHARGER OF AN IMPLANTABLE MEDICAL DEVICE

(71) Applicant: Boston Scientific Scimed, Inc,, Maple Grove, MN (US)

(72) Inventors: Matthew Philip Dion, Saint Francis, MN (US); Joey Chen, Stevenson Ranch, CA (US); Brian Thomas Swanson, Fridley, MN (US); Jason Lahr, Coon Rapids, MN (US); Lev Freidin, Simi Valley, CA (US); Keith R. Maile, New Brighton, MN (US)

(21) Appl. No.: 19/549,602

(22) Filed: Feb. 25, 2026

Related U.S. Application Data
(60) Provisional application No. 63/766,701, filed on Mar. 4, 2025.

(51) Int. Cl. H02J 50/12; A61F 2/00; A61F 2/26; A61N 1/36; A61N 1/378; H02J 50/90

(52) U.S. Cl. CPC H02J 50/12; A61F 2/0036; A61F 2/26; A61N 1/3605; A61N 1/3787; A61F 2250/0001; H02J 50/90

(57) ABSTRACT

A method of controlling an external charger configured to charge a battery of an implantable medical device is provided in which the method includes: providing an alternating current (AC) waveform having a driving frequency from an electrical circuit to a transmission coil to cause the transmission coil to generate a time-varying magnetic field, the electrical circuit having a resonant frequency; and controlling one or more switches to switch one or more capacitors into the electrical circuit to maintain the resonant frequency of the circuit equal to the driving frequency while the AC waveform is provided from the electrical circuit to the transmission coil.

### CROSS-REFERENCE TO RELATED APPLICATION

This application claims priority to U.S. Provisional Patent Application No. 63/766,701, filed on Mar. 4, 2025, entitled “DIGITAL CONTROL AND ACTIVE TUNING FOR A WIRELESS CHARGER OF AN IMPLANTABLE MEDICAL DEVICE”, the disclosure of which is incorporated by reference herein in its entirety.

### TECHNICAL FIELD

This disclosure relates generally to wireless charging devices and, in particular, to digital control and active tuning for a wireless charger of an implantable medical device.

### BACKGROUND

A medical device may be implanted into the body of a patient, and the medical device may have electronic circuitry powered by a battery source that is rechargeable. To recharge the battery source, a transmitter member of the external charger (e.g., outside of the body) may be aligned with a receiver member of the medical device (e.g., inside of the body), so that electrical energy can be transmitted from the external charger to the implantable medical device through inductive coupling of the energy from the external charger to the implantable medical device. Efficient transmission of electrical energy from the external charger to the implantable medical device depends on the transmission circuit of the external charger and the receiving circuit of the implantable medical device being tuned to a common resonant frequency. When the transmission circuit or the receiving circuit is not tuned to the resonant frequency, then the efficiency of energy transfer may suffer, such that a battery source in the implantable medical device cannot be sufficiently charged, or such that charging of the battery source requires a longer time than desired, or such that energy is wasted during the charging of the battery source.

### SUMMARY

In some aspects, the techniques described herein relate to an external charger configured for charging a battery of an implantable medical device. The external charger includes: a transmission coil configured for generating a time-varying magnetic field with a driving frequency; an electrical circuit for providing an alternating current (AC) waveform to the transmission coil, where providing the AC waveform to the transmission coil causes the transmission coil to generate the time-varying magnetic field, and where the electrical circuit has a resonant frequency; one or more capacitors; one or more switches configured to switch the one or more capacitors into the electrical circuit; and a controller configured for controlling the one or more switches to switch the one or more capacitors into the circuit to maintain the resonant frequency of the circuit equal to the driving frequency.

Implementations can include one or more of the following features, alone, or in any combination with each other.

For example, the one or more capacitors can include at least a first capacitor having a capacitance, C, a second capacitor having a capacitance, 2C, and a third capacitor having a capacitance, 4C, the first, second, and third capacitors being arranged in parallel with each other, wherein the one or more switches includes a first switch connected in series with the first capacitor, a second switch connected in series with the second capacitor and a third switch connected in series with the third capacitor, wherein the controller is configured to control the switches to add a capacitance of C, 2C, 3C, 4C, 5C, 6C or 7C to the electrical circuit.

In another example, the external charger can further include a current sensing circuit configured to sense a current provided to the transmission coil, wherein the controller is configured to determine the capacitance to add to the electrical circuit based on the current sensed by the sensing circuit.

In another example, the controller can be configured to: control the switches to add different discrete capacitances to the electrical circuit, receive signals from the current sensing circuit indicating a current provided to the transmission coil when the different discrete capacitances are added to the circuit, execute an algorithm, based on the received signals, to determine a capacitance that maximizes the current provided to the transmission coil, and control the switches to add the capacitance that maximizes the current provided to the transmission coil to maintain the resonant frequency equal to the driving frequency.

In another example, the charger can be configured to: receive a signal from the current sensing circuit indicating a current provided to the transmission coil is greater than a threshold current, and when the current provided to the transmission coil is greater than the threshold current; control the switches to add different discrete capacitances to the electrical circuit, receive signals from the current sensing circuit indicating a current provided to the transmission coil when the different discrete capacitances are added to the circuit, execute an algorithm, based on the received signals, to determine a capacitance that maximizes the current provided to the transmission coil, and control the switches to add the capacitance that maximizes the current provided to the transmission coil to maintain the resonant frequency equal to the driving frequency.

In another example, the algorithm includes a Fibonacci search algorithm.

In another example, the one or more capacitors can include at least a fourth capacitor having a capacitance, C₀, and the one or more switches can include a fourth switch connected in series with the fourth capacitor, where the controller is configured to control the fourth switch to periodically switch the fourth switch between a closed state and an open state, the switching occurring during dead time periods of the electrical circuit that provides the alternating current waveform to the transmission coil, the switching occurring with a duty cycle (DC) and causing a time-averaged capacitance equal to DC* C₀ to be added to the electrical circuit.

In another example, the duty cycle can be determined by controlling a length of time with which the fourth switch is in a closed state during the periodic switching of the fourth switch.

In another example, the duty cycle can be determined by controlling a frequency with which the fourth switch is switched between the open state and the closed state during the periodic switching of the fourth switch.

In another example, the implantable medical device includes an inflatable penile prosthesis, a urinary control device, or a neurostimulation medical implant.

In some aspects, the techniques described herein relate to a method of controlling an external charger configured to charge a battery of an implantable medical device, where the method includes: providing an alternating current (AC) waveform having a driving frequency from an electrical circuit to a transmission coil to cause the transmission coil to generate a time-varying magnetic field, the electrical circuit having a resonant frequency; and controlling one or more switches to switch one or more capacitors into the electrical circuit to maintain the resonant frequency of the circuit equal to the driving frequency while the AC waveform is provided from the electrical circuit to the transmission coil.

Implementations can include one or more of the following features, alone, or in any combination with each other.

For example, the one or more capacitors can include at least a first capacitor having a capacitance, C, a second capacitor having a capacitance, 2C, and a third capacitor having a capacitance, 4C, the first, second, and third capacitors being arranged in parallel with each other, wherein the one or more switches includes a first switch connected in series with the first capacitor, a second switch connected in series with the second capacitor and a third switch connected in series with the third capacitor, and the method further including: controlling the switches to add a capacitance of C, 2C, 3C, 4C, 5C, 6C or 7C to the electrical circuit.

In another example, the method can further include sensing a current provided to the transmission coil; and controlling the switches to add the capacitance of C, 2C, 3C, 4C, 5C, 6C or 7C to the electrical circuit based on the sensed current.

In another example, the method can further include controlling the one or more switches to add different discrete capacitances to the electrical circuit; sensing the current provided to the transmission coil when the different discrete capacitances are added to the circuit; executing an algorithm, based on the sensed current, to determine a capacitance that maximizes the current provided to the transmission coil; and controlling the switches to add the capacitance that maximizes the current provided to the transmission coil to maintain the resonant frequency equal to the driving frequency.

In another example, the method can further include: sensing that a current provided to the transmission coil is greater than a threshold current, and when the current provided to the transmission coil is greater than the threshold current; controlling the switches to add different discrete capacitances to the electrical circuit; sensing the current provided to the transmission coil when the different discrete capacitances are added to the circuit; executing an algorithm, based on the sensed current, to determine a capacitance that maximizes the current provided to the transmission coil; and controlling the switches to add the capacitance that maximizes the current provided to the transmission coil to maintain the resonant frequency equal to the driving frequency.

In another example, the algorithm can include a Fibonacci search algorithm.

In another example, the one or more capacitors can include at least a fourth capacitor having a capacitance, C₀, wherein the one or more switches includes a fourth switch connected in series with the fourth capacitor, and the method can further include: controlling the fourth switch to periodically switch the fourth switch between a closed state and an open state, the switching occurring during dead time periods of the electrical circuit that provides the alternating current waveform to the transmission coil, the switching occurring with a duty cycle (DC) and causing a time-averaged capacitance equal to DC* C₀ to be added to the electrical circuit.

In another example, the duty cycle can be determined by controlling a length of time with which the fourth switch is in a closed state during the periodic switching of the fourth switch.

In another example, the duty cycle can be determined by controlling a frequency with which the fourth switch is switched between the open state and the closed state during the periodic switching of the fourth switch.

In another example, the implantable medical device can include an inflatable penile prosthesis, a urinary control device, or a neurostimulation medical implant.

### BRIEF DESCRIPTION OF THE DRAWINGS

FIG. 1A is a block diagram of a medical device and a power transmission device with an alignment detector for detecting an alignment value between the medical device and the power transmission device.

FIG. 1B illustrates an example of a power transmission device.

FIG. 1C illustrates a graph depicting values of a coil ratio for increasing distance between the medical device and the power transmission device.

FIG. 1D illustrates a graph depicting increasing values of a coil ratio for increasing values of an inner sense voltage according to an aspect.

FIG. 1E illustrates an example of transmitting load-shift keying (LSK) data based on voltage and/or current readings in a medical device according to an aspect.

FIG. 1F illustrates an example of LSK data indicating an alignment level with an external charger according to an aspect.

FIG. 2 is a schematic diagram of an example system for transmitting power from an external charger to an implantable medical device.

FIG. 3 is a schematic diagram of an electrical circuit for transmitting power from an external charger to an implantable medical device.

FIG. 4 is a schematic diagram of an example electrical circuit for transmitting power from an external charger to an implantable medical device.

FIG. 5A illustrates an example implantable fluid-operated inflatable device in the form of an example inflatable penile prosthesis.

FIG. 5B illustrates a urinary control device having an electronic pump assembly according to an aspect.

FIGS. 6A and 6B are schematic diagrams of example implantable neurostimulators that can be charged by an external power transmission device

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the implementations in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having,” as used herein, are defined as comprising (i.e., open transition). The term “coupled” or “moveably coupled,” as used herein, is defined as connected, although not necessarily directly and mechanically.

In general, the implementations are directed to bodily implants and/or external chargers configured to wireless charge the bodily implants. The term patient or user may be used to describe a person who benefits from the medical device or the methods disclosed in the present disclosure. For example, the patient can be a person whose body is implanted with the medical device or the method disclosed for operating the medical device by the present disclosure.

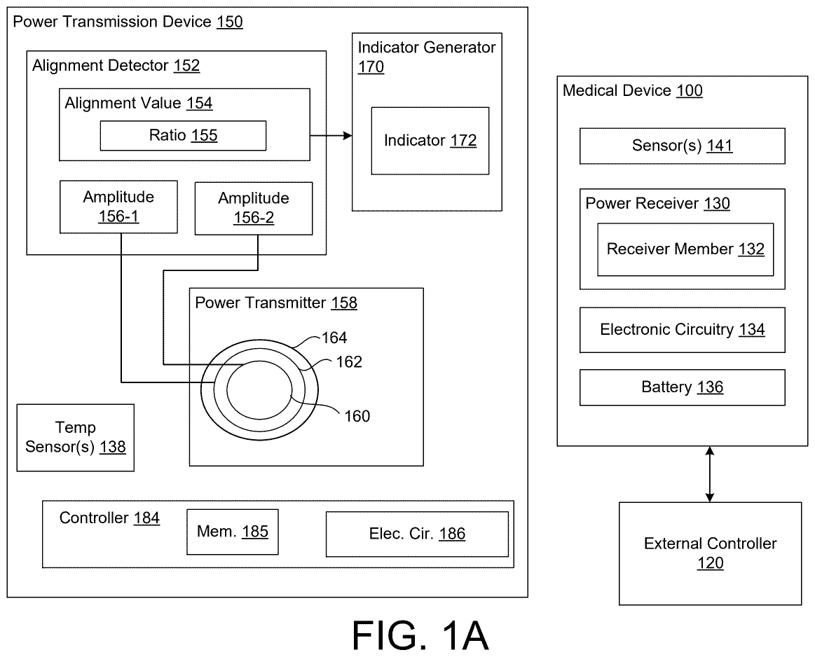

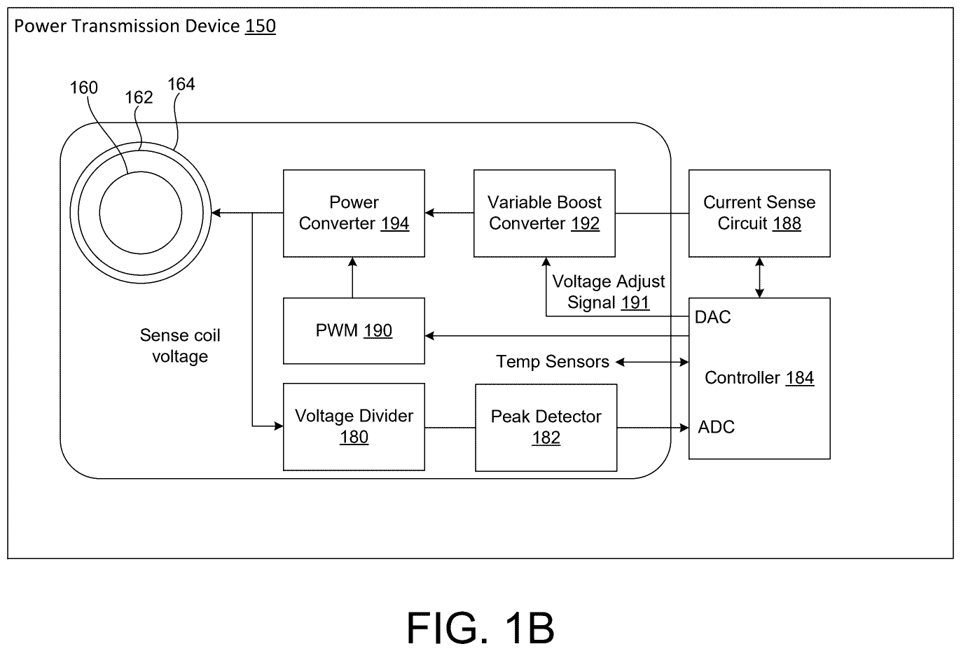

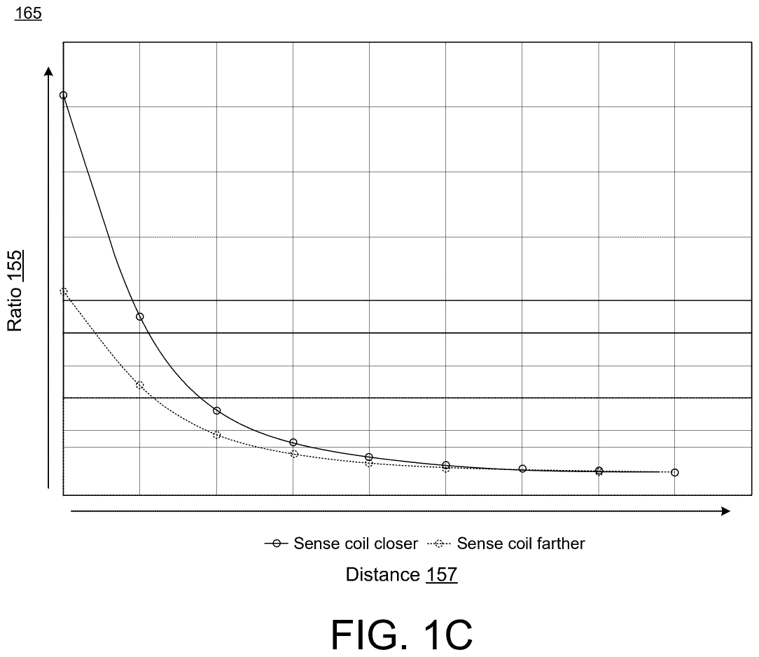

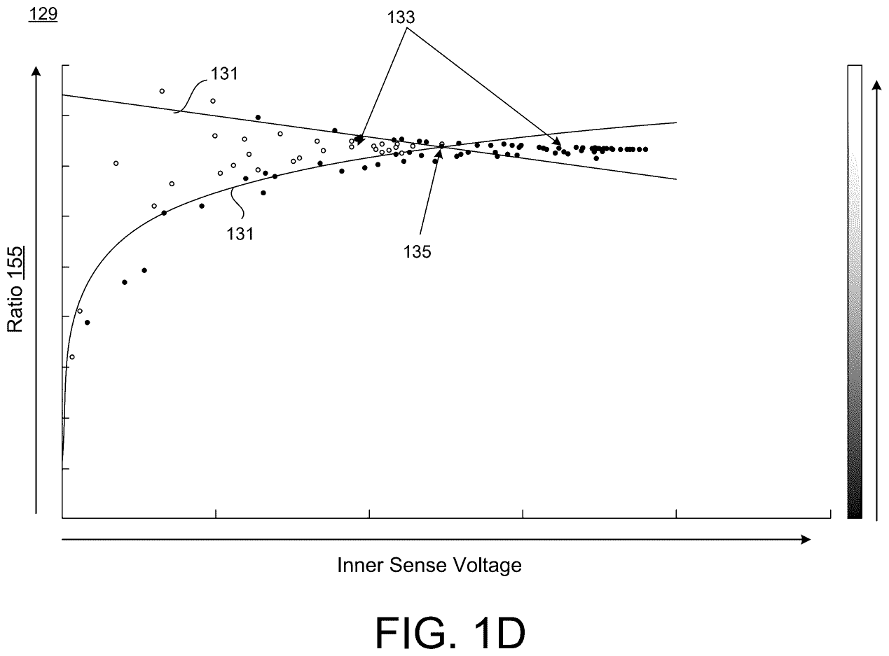

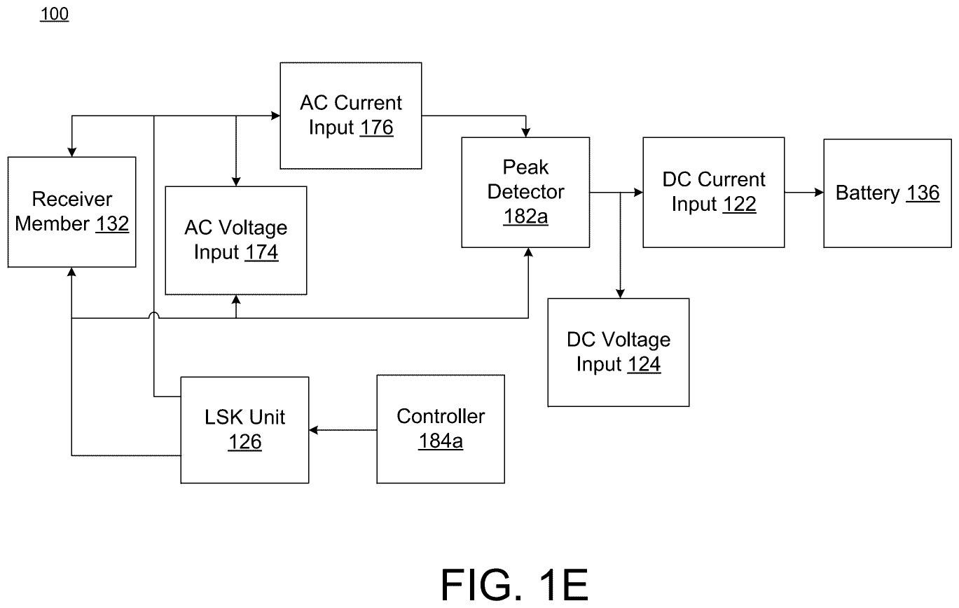

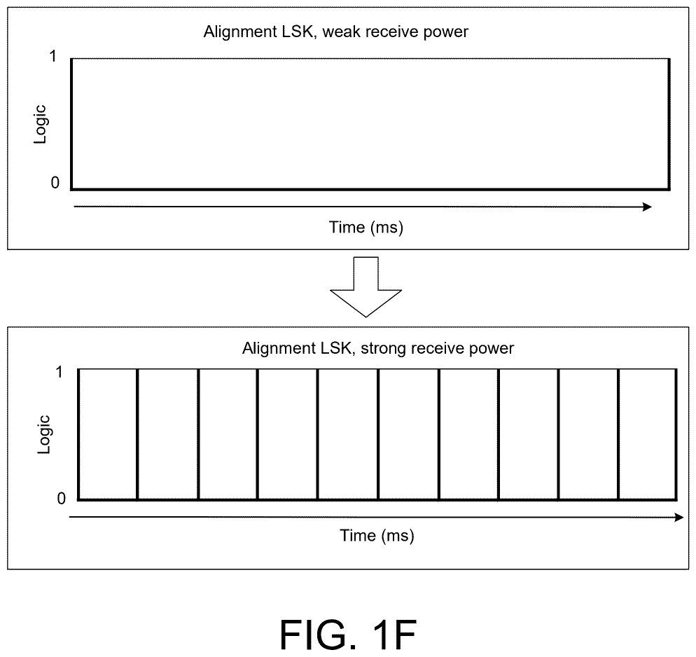

FIG. 1A is a schematic block diagram of an implantable medical device 100 and a power transmission device 150. FIG. 1B is a schematic block diagram of the power transmission device. The medical device 100 can be implanted into the body of a patient. The power transmission device 150 wirelessly charges (e.g., transfers energy to) the medical device 100 while the medical device 100 is implanted in the body of the patient. The transferred energy may charge a battery 136 of the medical device 100 and/or power electronic circuitry 134 of the medical device 100. In some examples, the power transmission device 150 is referred to as an external charger. In some examples, the power transmission device 150 is referred to as a wireless charger (e.g., power is delivered to the medical device 100 without the use of wires or cords between the medical device 100 and the power transmission device 150).

The power transmission device 150 includes a power transmitter 158 configured to generate an alternating current (AC) in a transmitter coil, which causes a time-varying magnetic field to wirelessly transfer energy to receiving coil of a power receiver 130 of the medical device 100. The time-varying magnetic field induces an alternating current in the receiving coil, and the current is used to charge a battery 136 of the medical device 100. In some examples, the power transmitter 158 is referred to as a charging pad (e.g., an external charging pad). The power transmitter 158 includes a transmitter member 164. In some examples, the transmitter member 164 is referred to as a transmitter coil or a charge coil. The power receiver 130 includes a receiver member 132. In some examples, the receiver member 132 is referred to as a receiving coil. An alternating current is excited in the transmitter coil 164, such that a magnetic field is emitted and propagated outwardly from the transmitter member 164, and, when the receiver member 132 is within range and in close proximity, the changing magnetic field induces a current in the receiver member 132. In some examples, the transmitter member 164 may have a spiral pattern (e.g., a flat spiral pattern). For increased power transfer performance, the transmitter member 164 and the receiver member 132 may be positioned within a threshold distance and aligned, to efficiently couple the magnetic flux from the transmitter coil 164 to the receiver member 132.

In some examples, alignment of the transmitter member 164 and the receiver member 132 may refer to the physical positioning of the transmitter member 164 to the receiver member 132 such that a first axis that extends through a center of the transmitter member 164 is within a threshold distance of a second axis that extends through a center of the receiver member 132. In some examples, the alignment of the transmitter member 164 and the receiver member 132 may refer to the strength of the magnetic field coupling such as the amount of magnetic flux generated by the transmitter member 164 that passes through the receiver member 132. In some examples, the more magnetic flux that is intercepted by the receiver member 132, the stronger the coupling and the more efficient the power transfer.

The power transmitter 158 includes an inner sensor member 160 and an outer sensor member 162. In some examples, the inner sensor member 160 is referred to as an inner sense coil. In some examples, the outer sensor member 162 is referred to as an outer sense coil. In some examples, the outer sensor member 162 is a size (e.g., a diameter) that is less than a size (e.g., diameter) of the transmitter member 164. The inner sensor member 160 may have a diameter that is smaller than the diameter of the outer sensor member 162.

In some examples, the inner sensor member 160 is a first printed circuit board (PCB) trace, and the outer sensor member 162 is a second PCB trace. In some examples, each of the inner sensor member 160 and the outer sensor member 162 may detect a magnetic field and/or a back electromotive force (EMF) caused by eddy currents, which cancels a portion of the magnetic field in a center portion (e.g., the center) of the power transmitter 158. For example, when the medical device 100 is placed within the range of the transmitter field (e.g., the magnetic field) of the power transmitter 158, the changing magnetic field can induce eddy currents.

In some examples, the inner sensor member 160 includes a circular (conductive) portion. In some examples, the outer sensor member 162 includes a circular (conductive) portion. In some examples, the inner sensor member 160 includes a spiral circular pattern with a flat shape. In some examples, the outer sensor member 162 includes a spiral circular pattern with a flat shape. In some examples, the inner sensor member 160 and/or the outer sensor member 162 may have the same shape and/or structure. In some examples, the inner sensor member 160 and/or the outer sensor member 162 may have different shapes and/or structures. In some examples, the inner sensor member 160 and/or the outer sensor member 162 may have a cloverleaf shape. In some examples, the inner sensor member 160 and/or the outer sensor member 162 may have a butterfly shape.

The power transmission device 150 includes an alignment detector 152 configured to generate an alignment value 154. The alignment value 154 may represent the degree of alignment between the receiver member 132 and the transmitter member 164. In some examples, the transmitter member 164 is referred to as a charge coil or a charging coil. In some examples, the alignment value 154 includes a ratio 155 of an amplitude 156-1 of the inner sensor member 160 and an amplitude 156-2 of the outer sensor member 162. In some examples, the ratio 155 is referred to as a sense amplitude ratio (e.g., a sense coil amplitude ratio). In some examples, the ratio 155 increases as the transmitter member 164 becomes closer and/or centered with respect to the receiver member 132.

The alignment detector 152 may sense (e.g., detect) an amplitude 156-1 of the voltage on the inner sensor member 160 and may sense (e.g., detect) an amplitude 156-2 of the voltage on the outer sensor member 162. In some examples, the alignment detector 152 detects the amplitude 156-1 and the amplitude 156-2 according to a sampling rate. In some examples, by using the amplitude 156-1 and the amplitude 156-2, a lower sampling rate can be used (as compared to some conventional approaches), thereby reducing the amount of processing power. The alignment detector 152 may compute the alignment value 154 as the ratio 155 of the amplitude 156-1 and the amplitude 156-2. In some examples, the ratio 155 between the amplitude 156-1 and the amplitude 156-2 increases as the charge coil (e.g., the transmitter member 164) gets closer and/or better centered with respect to the receiver member 132.

In some examples, the power transmission device 150 includes an indicator generator 170 configured to generate an indicator 172 according to the alignment value 154. The indicator 172 may provide feedback to the user about the alignment of the power transmission device 150 to the medical device 100. In some examples, the indicator 172 is a visual indicator. In some examples, the indicator 172 is an audio indicator. In some examples, the indicator 172 includes a visual and audio indicator. The indicator 172 may change according to the alignment values 154 so that the user can determine whether movement of the power transmission device 150 causes the power transmitter 158 to be better aligned with the power receiver 130 of the medical device 100.

Referring to FIG. 1B, in some implementations, the power transmission device 150 may include a voltage divider 180, a peak detector 182, and a controller 184. In some examples, the alignment detector 152 includes the voltage divider 180, the peak detector 182, and the controller 184. The voltage divider 180 may include two resistors connected in series across the inner sensor member 160 and the outer sensor member 162.

In some examples, the peak detector 182 may rectify the sense voltages (e.g., sense coil voltages). The peak detector 182 may convert the voltage signal to a voltage waveform signal (e.g., with an envelope level or a peak level). The peak detector 182 receives a first sense coil voltage at the inner sensor member 160 and a second sense coil voltage at the outer sensor member 162. The peak detector 182 may generate a first voltage waveform based on the first sense coil voltage and a second voltage waveform based on the second sense coil voltage. In some examples, the first voltage waveform is an analog voltage signal that includes the peak amplitude at the inner sensor member 160. In some examples, the second voltage waveform is an analog voltage signal that includes the peak amplitude at the outer sensor member 162.

In some examples, the first voltage waveform may represent an envelope signal at the inner sensor member 160. In some examples, the second voltage waveform may represent an envelope signal at the outer sensor member 162. In some examples, the first voltage waveform may represent a rectified signal at the inner sensor member 160. In some examples, the second voltage waveform may represent a rectified signal at the outer sensor member 162.

The peak detector 182 may include one or more diodes. In some examples, the peak detector 182 may include one or more capacitors. In some examples, the peak detector 182 may include one or more diodes and one or more capacitors. In some examples, the peak detector 182 includes a half-wave rectifier circuit (e.g., a single diode). In some examples, the peak detector 182 includes a full-wave rectifier (e.g., four or more diodes arranged in a bridge circuit).

In some examples, the controller 184 includes a microcontroller. In some examples, the controller 184 includes an integrated circuit. In some examples, the controller 184 includes an analog-to-digital converter and/or a digital-to-analog converter. In some examples, the controller 184 includes a memory 185 for storing machine-readable instructions and/or data. In some implementations, the controller 184 includes electrical circuitry (e.g., a processor, and integrated circuit, and ASIC, etc.) 186 configured for executing the stored machine-readable instructions and/or processing the stored data. In some examples, the controller 184 includes one or more interfaces configured to communicate with sensors (e.g., temperature sensor 138) and/or other components such as a current sense circuit 188.

In some examples, the controller 184 may sample the first and second voltage waveforms according to a sample rate and generate, for each sample, a first digital value representing the amplitude 156-1 at the inner sensor member 160 and a second digital value representing the amplitude 156-2 at the outer sensor member 162. The controller 184 may compute a ratio (e.g., a ratio 155) of the first digital value representing the amplitude 156-1 at the inner sensor member 160 and the second digital value representing the amplitude 156-2 at the outer sensor member 162. In some examples, the controller 184 may store the ratio 155 for each sample (or a portion of the samples). In some examples, once rectified, the sense coil amplitudes (e.g., amplitude 156-1, amplitude 156-2) may be read by the controller 184 at a sample rate (e.g., less than 100 times per second, less than 50 times per second, less than 15 times per second, etc.).

In some examples, the controller 184 receives a temperature of the power transmitter 158 (e.g., a temperature of the transmitter member 164) from a temperature sensor 138 on the power transmission device 150. In some examples, the controller 184 receives the temperature via an inter-integrated circuit (I2C) transmission protocol. In some examples, the controller 184 may adjust the power (e.g., voltage, current) transmitted by the power transmitter 158 based on the temperature sensor 138. In some examples, in response to the temperature being greater than a threshold level, the controller 184 may reduce or stop the transmission of energy via the power transmitter 158.

In some examples, the power transmission device 150 includes a current sense circuit 188 configured to detect an output voltage of the transmitter member 164. In some examples, the current sense circuit 188 may detect an analog voltage signal proportional to a voltage source (e.g., VBAT). In some examples, the power transmission device 150 includes a variable boost converter 192 configured to adjust the voltage from the voltage source according to a voltage adjust signal 191 (e.g., an analog control signal) from the controller 184. In some examples, the variable boost converter 192 includes an analog-to-analog voltage converter. The controller 184 may generate an analog control signal (e.g., a voltage adjust signal 191) using a digital-to-analog converter, and the variable boost converter 192 may increase (or decrease) the voltage according to the voltage adjust signal 191. In some examples, the voltage adjust signal 191 is referred to as a reference signal.

In some examples, the power transmission device 150 includes a pulse width modulator (PWM) 190 configured to control a duty signal based on a control signal from the controller 184. The PWM 190 may output a control signal that switches between a high state and a low state at a fixed frequency. In some examples, the PWM 190 includes a high-resolution PWM. The power transmission device 150 may include a power converter 194 that receives the voltage from the variable boost converter 192 and generates a voltage that is applied to the transmitter member 164 according to the control signal generated by the PWM 190. In some examples, the power converter 194 includes a half-bridge circuit. In some examples, the power converter 194 includes a full-bridge circuit. In some examples, the power converter 194 may include switching elements (e.g., transistors) (e.g., Metal-Oxide-Semiconductor Field-Effect Transistors (MOSFETs), configured in a bridge arrangement. These transistors may operate as high-speed electronic switches that can be turned on and off to provide an alternating current waveform to the transmitter member 162.

In some examples, the medical device 100 includes one or more sensors 141. In some examples, the sensor(s) 141 includes a magnetic field sensor (e.g., a Hall effect sensor). In some examples, the transmitter member 164 includes a magnet. In some examples, the transmitter member 164 includes a static magnet. In some examples the sensor 141 (e.g., the magnetic field sensor) (e.g., Hall effect sensor) may sense the strength of the magnetic field of the static magnet in the transmitter member 164. The strength of the magnetic field may provide an indication (e.g., the indicator 172) of the alignment of the transmitter member 164 and the receiver member 132. In some examples, the strength of the magnetic field may be communicated from the medical device 100 to the user.

In some examples, the electronic circuitry 134 may include a telemetry communication engine (e.g., LSK unit 126) configured to communicate the sensed magnitude strength (e.g., the indicator 172) to an external controller 120, which can display visual feedback of the alignment between the transmitter member 164 and the receiver member 132. The external controller 120 may be a device that can control one or more functions of the medical device 100. In some examples, the medical device 100 communicates the indicator 172 to another device besides an external controller 120 such as a user device (e.g., an application on a smartphone, a computer, or other computing device).

In some examples, the sensor(s) 141 may include one or more temperature sensors that measure the temperature of the medical device 100 (or a portion thereof). In some examples, the temperature sensor is located in an electronic pump of the medical device 100. The alignment detector 152 may use the temperature data of the temperature sensor to generate an indicator 172 about the alignment of the transmitter member 164 with the receiver member 132. Charging will heat the medical device 100. If the transmitter member 164 and the receiver member 132 are out of alignment, the temperature data may indicate uneven temperature data.

In some examples, power transmission device 150 includes an inertial sensor such as a gyroscopic, an accelerometer, and/or an inertial measurement unit (IMU). The inertial sensor may determine a positional data of the power transmission device 150 and use that positional data to determine its location relative to the receiver member 132. The power transmission device 150 may determine an indicator 172 based on the positional data.

FIG. 1C illustrates a graph 165 depicting a ratio 155 between voltages on the outer sensor member 162 and the inner sensor member 160 as a function of a distance 157 between the transmitter member 164 (e.g., transmitter coil) and the receiver member 132 (e.g., receiver coil). In some examples, the ratio 155 between the outer sensor member 162 and the inner sensor member 160 increases as the transmitter member is brought closer, and/or centered relative to, the receiver member 132. In some examples, the transmitted power can be varied, and the ratio 155 is independent of the transmitted power of the transmitter member 164.

FIG. 1D illustrates a graph 129 depicting values of a coil ratio 155 as a function of an inner sense voltage. In some examples, the alignment detector 152 may define an alignment area 133. The alignment area 133 may be defined by one or more alignment boundaries determined by one or more curves 131 from a curve-fitting algorithm. The curves 131 determined by the curve-fitting algorithm may provide a two-dimensional (2D) map of ratios 155 versus an inner sense voltage. In some examples, to determine alignment of the external charger, the alignment detector 152 may use the ratio 155 and the inner sensor voltage to determine whether that data point falls within a predetermined alignment area 133. The closer the data point is to an intersection 135 of the curves 131, the better alignment of transmitter member 164 with the receiver member 132.

FIGS. 1E and 1F illustrate an example of transmitting load-shift keying (LSK) data based on voltage and/or current readings in a medical device according to an aspect. FIG. 1F illustrates an alignment LSK for weak and strong received power. In some examples, instead of the power transmission device 150 computing an indicator 172 that represents a level of alignment between the power transmission device 150 and the medical device 100, the medical device 100 may detect voltage and/or current readings in the receiver member 132 and use those readings to generate an indicator 172, which is communicated back to the user using LSK. In some examples, the medical device 100 may measure input voltage and/or current to determine the power received by an external charger. The medical device 100 may transmit backscatter LSK data to indicate an optimal alignment. In some examples, the pulse width could change to indicate alignment. In some examples, the medical device 100 may transmit the backscatter LSK data with the determined power to the external charger.

For example, the medical device 100 may detect an AC voltage input 174 and/or AC current input 176 of the receiver member 132. In some examples, the medical device 100 includes a peak detector 182a configured to compute a DC current input 122 and/or a DC voltage input 124. In some examples, the DC current input 122 is provided to the battery 136. In some examples, a controller 184a on the medical device may compute the power from the measured DC current input 122 and/or the DC voltage input 124 and provide the calculated power to a LSK unit 126 that computes LSK data with the computed power, which can be transmitted by the receiver member 132. In some examples, the LSK unit 126 encodes the detected power and transmits the data to another device, which may be the external charger.

In some examples, the medical device 100 is an implantable fluid-operated inflatable device, which may include a fluid reservoir, an inflatable member, and an electronic control system.

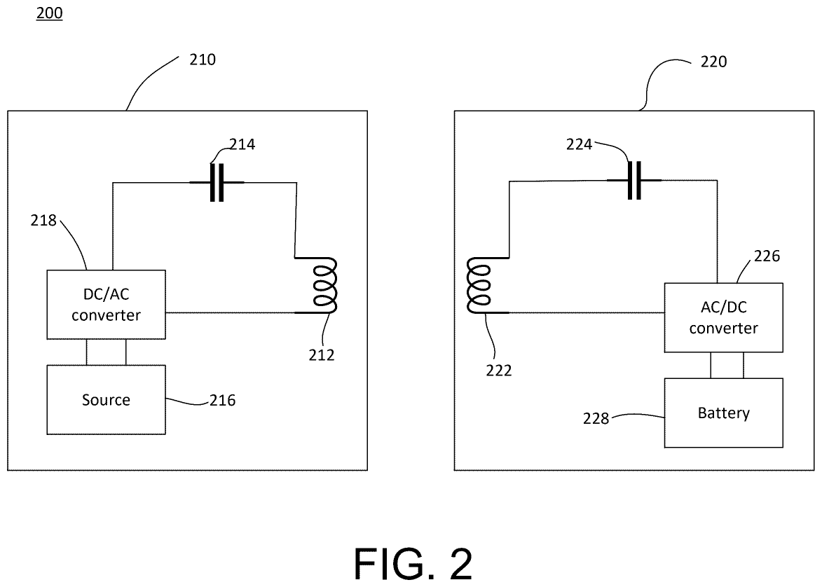

FIG. 2 is a schematic diagram of an example system 200 for transmitting power from an external charger 210 to an implantable medical device 220, where the external charger 210 transmits electrical power from a transmission coil 212 in the external charger, which has a first inductance, L₁, to a receiving coil 222 in the implantable medical device, which has a second inductance, L₂. The external charger 210 includes a capacitor 214 that has a capacitance, C₁, and the implantable medical device 220 includes a capacitor 224 that has a capacitance, C₂. The transmission coil 212 and the capacitor 214 of the external charger form, or are part of, a resonant circuit that has a resonant frequency f₁, where the resonant frequency is the frequency at which the inductive and capacitive reactance in the circuit cancel each other out, resulting in a purely resistive impedance. The receiving coil 222 and the capacitor 224 of the implantable medical device form, or part of, a resonant circuit that has a resonant frequency f₂. For the series-resonant circuit of the external charger 210, the resonant frequency can be defined as a function of the inductance of the transmission coil 212 and the capacitance of the capacitor 214 as:

$${f}_{1} = \frac{1}{2 π \sqrt{{L}_{1} {C}_{1}}} . \qquad ( 1 )$$

For the series-resonant circuit of the implantable medical device 220, the resonant frequency can be defined as a function of the inductance of the receiving coil 222 and the capacitance of the capacitor 224 as:

$${f}_{2} = \frac{1}{2 π \sqrt{{L}_{2} {C}_{2}}} . \qquad ( 2 )$$

The circuits of the external charger 210 and of the implantable medical device 220 each have a quality factor (Q) that is a measure of the sharpness of the resonance and determines the bandwidth over which the circuit can operate effectively. A high-Q circuit has a narrow bandwidth and can store energy efficiently compared to a low-Q circuit.

The external charger 210 further includes a power source 216, for example, a battery, that is configured to provide a direct current (DC) current to a DC/AC converter 218. The DC/AC converter 218 converts the power supplied by the power source 216 into an alternating current (AC) having a drive frequency fa. The drive frequency can be selected to conform to one or more standards for transmitting power to the implantable medical device 220. For example, the drive frequency can conform to an Industrial, Scientific, and Medical (ISM) standard and can be, for example, 6.78 MHz or 13.56 MHz. The electrical components of the external charger 210 and the implantable medical device 220 are shown in simplified form and other configurations are also possible. For example, while the external charger 210 and the implantable medical device 220 are shown as including series resonant circuits with a capacitor in series with a transmission/receiving coil, other implementations, including, for example, parallel-series resonant and series-parallel resonant circuits using multiple capacitors in series with, and in parallel to, the transmission/receiving coil, are also possible.

Electrical power can be transferred from the external charger 210 to the implantable medical device 220 when the resonant frequencies of the two devices are approximately equal, for example, when the frequencies differ by less than the sum of the bandwidths specified by the Q values of the two circuits. The AC power received by the receiving coil 222 of the implantable medical device can be converted into a DC current by an AC/DC converter 226 and used to charge a battery 228.

The circuits of the external charger 210 and of the implantable medical device can be designed such that their resonant frequencies are equal to the ISM drive frequency used to transfer power from the external charger to the implantable medical device. However, when the transmission coil 212 of the external charger 210 is brought into close proximity and alignment with the receiving coil 222 of the implantable medical device 220, mutual inductance between the two coils can cause the resonant frequency of the circuit of the external charger to shift from its unperturbed resonant frequency. For example, the mutual inductance between the two coils can lead to a lower effective inductance of the electrical circuit of the external charger, such that the resonant frequency of the perturbed circuit is higher than the resonant frequency of the unperturbed circuit.

Therefore, to maintain the drive frequency of the external charger 210 at the frequency specified by the ISM standard and to also maintain the resonance frequency of the circuit at the drive frequency, the circuit can be modified in response to the mutual inductance effects that are present when the transmission coil of the external charger is proximate to, and aligned with, the receiving coil of the implantable medical device. In some implementations, the capacitance of the electrical circuit of the external charger 210 can be adjusted in response to the mutual inductance due to the coupling between the transmission circuit and the receiving circuit.

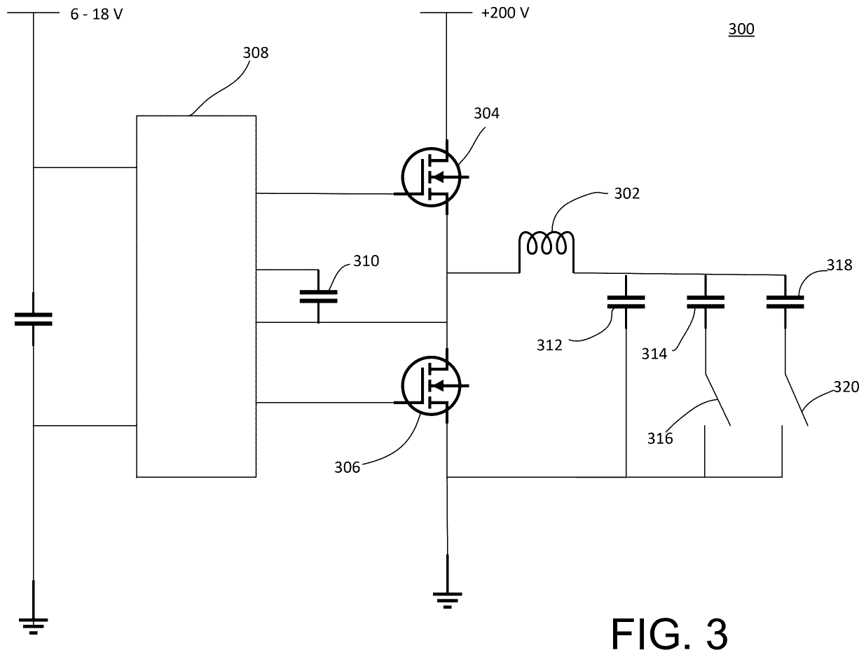

FIG. 3 is a schematic diagram of an electrical circuit 300 for transmitting power from an external charger to an implantable medical device. In some implementations, the circuit 300 can include an H-bridge circuit configured for providing an AC current to a transmission coil 302. The H-bridge circuit can include a first transistor 304 (e.g., a MOSFET) and a second transistor 306 (e.g., a MOSFET) connected in series between a high voltage and ground, with the transmission coil being connected to a node between the transistors. The first and second transistors 304, 304 can be alternately turned on by an integrated circuit controller 308 to switch the direction of current through the transmission coil 302. A bootstrap capacitor 310 is used to temporarily store charge and provide a higher voltage than the power supply to drive the gate of a high-side MOSFET 304, allowing it to turn on properly. Thus, the bootstrap capacitor bootstraps the voltage needed to activate the high-side MOSFET 304 by utilizing the stored charge when the low-side MOSFET 306 is on.

The circuit 300 includes a first capacitor 312 that can be permanently connected in series with the transmission coil 302. The capacitance, C, of the first capacitor 312 and the inductance, L, of the transmission coil 302 define an unperturbed resonant frequency, fᵤₚ, of the circuit according to:

$${f}_{up} = \frac{1}{2 π \sqrt{LC}} , \qquad ( 3 )$$

where the unperturbed frequency can correspond to the drive frequency of the circuit.

When the circuit 300 is used in an external charger to transmit electrical energy to an implantable medical device and mutual inductance between the transmission coil 302 and a receiving coil of the implantable medical device induces an increase in the resonant frequency, the resonant frequency can be adjusted by adding additional capacitance to the circuit 300, so as to maintain the resonant frequency of the circuit equal to the drive frequency of the circuit.

In some implementations, one or more second capacitors 314 can be switched into the circuit 300 in series with the transmission coil 302 by one or more switches 316. The one or more second capacitors 314 can be switched into the circuit 300 and connected continuously to the circuit for multiple cycles (e.g., for more than 100 cycles, more than 300 cycles, more than 1000 cycles, or more than 10,000 cycles) of the drive frequency, such that the one or more second capacitors 314, when switched into the circuit can be considered to provide a static change to the capacitance of the circuit. Thus, when mutual inductance between the transmission coil and the receiving coil causes the circuit 300 to have an effective inductance, Lₑ, that is less than the unperturbed inductance, L, the addition of the capacitance, C₂, of the one or more second capacitors can maintain the resonant frequency at the drive frequency according to:

$${f}_{res} = \frac{1}{2 π \sqrt{{L}_{e} ( C + {C}_{2} )}} , \qquad ( 4 )$$

In some implementations, one or more third capacitors 318 can be switched into the circuit 300 in series with the transmission coil 302 by one or more switches 320. The one or more third capacitors 318 having a capacitance, C₃, can be switched dynamically into the circuit 300, such that over a number of cycles of the drive frequency the one or more third capacitors 318 are repeatedly switched into the circuit with a duty cycle between 0% and 100%. Because of this, when mutual inductance between the transmission coil and the receiving coil causes the circuit 300 to have an effective inductance, Lₑ, that is less than the unperturbed inductance, L, the addition of the capacitance, C₃, that is dynamically-switched with a duty cycle, DC, between 0% and 100%, can maintain the resonant frequency at the drive frequency according to:

$${f}_{res} = \frac{1}{2 π \sqrt{{L}_{e} ( C + DC * {C}_{3} )}} , \qquad ( 4 )$$

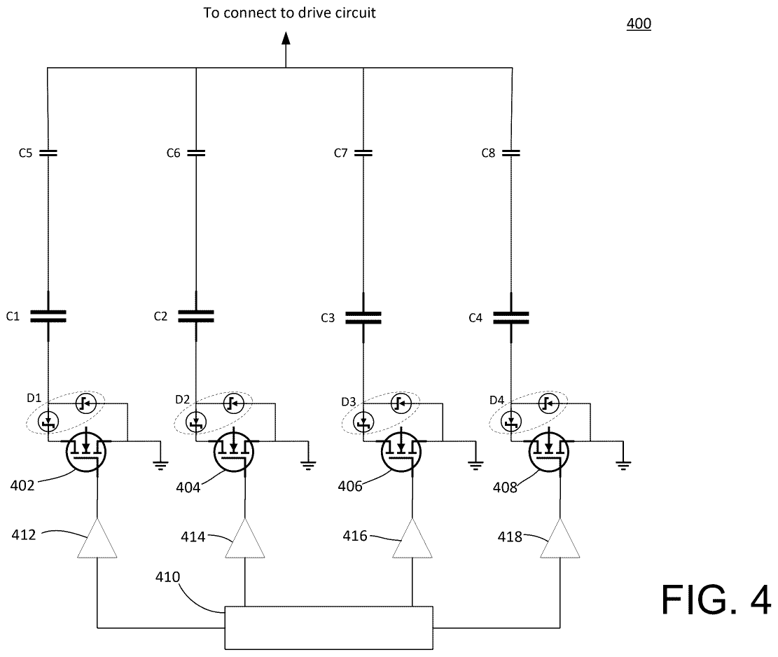

FIG. 4 is a schematic diagram of an example electrical circuit 400 for transmitting power from an external charger to an implantable medical device. The electrical circuit 400 can include, for example, details of the portion of the electrical circuit 300 that provides for static tuning of the capacitance of the transmission circuit to maintain a resonant frequency of the circuit 300 equal to a drive frequency of the circuit 300 in the presence of mutual inductance effects between the transmission circuit and a receiving circuit that receives energy from the transmission circuit.

In particular, the example circuit 400 includes details about an example implementation of the one or more second capacitors 314 and components for switching the one or more second capacitors into the circuit 300. The circuit 400 includes a plurality of capacitors C1, C2, C3, C4 arranged in parallel that can be used, individually, or in combination with each other, as the one or more second capacitors 314. Each capacitor C1, C2, C3, C4 can be switched between a connected state and an unconnected state by an associated switch 402, 404, 406, 408. In some implementations, the switches 402, 404, 406, 408 can be implemented as a MOSFET whose state is controlled by a signal received from a controller 410. In some implementations, a diode can be connected between the source and the drain of the MOSFET to protect the MOSFET against transient currents that could damage the MOSFET.

The capacitances of capacitors C1, C2, C3, C4 can have different values, the sum of which can be sufficient to adjust the resonant frequency of the circuit 300 to account for the maximum expected mutual inductance in the circuit, so that the resonant frequency can remain at the drive frequency according to equation (4). In some implementations, the capacitances of the individual capacitors C1, C2, C3, C4 can have different values, such that different combinations of the capacitors can provide a relatively-fine grained range of values for the capacitance that is added to the circuit 300 due to the connection of the one or more capacitors 314 to the circuit. For example, the capacitance of C2 can be twice the capacitance of C1, and the capacitance of C3 can be four times the capacitance of C1, and the capacitance of C4 can be eight times the capacitance of C1. Thus, if the capacitance of C1 is equal to C, the capacitance of C2 can be 2C, the capacitance of C3 can be 4C, and the capacitance of C4 can be 8C. With these values of the capacitances of the capacitors C1, C2, C3, C4 and with individual control of switches 402, 404, 406, 408 associated with the capacitors, the capacitance added to the circuit 300 can be digitally adjusted in increments of the capacitance of C1 over a range of 0 to 15 times the value of the capacitance of C1.

In some implementations, a high voltage capacitor C5, C6, C7, C8 can be connected in series with each of the respective capacitors C1, C2, C3, C4, so that the capacitors C1, C2, C3, C4 can operate at a relatively low voltage. The capacitances of the capacitors C5, C6, C7, C8 can be related to each other by the same ratios by which the capacitors C1, C2, C3, C4 are related to each other. For example, the capacitance of C5 can by 0.125 times the capacitance of C1; the capacitance of C6 can by 0.125 times the capacitance of C2; the capacitance of C7 can by 0.125 times the capacitance of C3; and the capacitance of C8 can by 0.125 times the capacitance of C4.

The controller 410 can control the state of the switches 402, 404, 406, 408 by providing digital signals to the switches. In some implementations, the signal provided to a switch 402, 404, 406, 408 (e.g., provided to the gate of a MOSFET) can be provided through buffers 412, 414, 416, 418. The buffers can be relatively low speed as compared with the frequency of the AC current in the transmission coil 302, because the capacitors C1, C2, C3, C4 are intended to provide a relatively static correction to the capacitance of the circuit 300.

In some implementations, when a MOSFET switch 402, 404, 406, 408 transitions from a closed (conductive) state to an open (non-conductive) state, its intrinsic output capacitance (Cₒₛₛ), including drain-to-source capacitance, can provide an unintended AC path from the associated capacitor to ground. This parasitic path causes the associated capacitor to remain effectively referenced to ground for a finite period of time, which can degrade the performance of the circuit. To mitigate this effect due to the parasitic capacitance of the switches, a diode rectifiers D1, D2, D3, and D4 can be connected in series, respectively, between the capacitors C1, C2, C3, C4 and ground, in parallel with respective MOSFET switches 402, 404, 406, 408, such that the MOSFET switch and the diode are arranged in a current path to ground when the MOSFET switch is closed, and such that, when the MOSFET switch is disconnected and changed to an open state, the diode is reverse-biased and isolates the associated capacitor from ground except for the diode's substantially smaller junction capacitance. By selecting a diode with low reverse capacitance (e.g., a high-speed silicon, Schottky, or PIN device appropriate to the operating frequency and voltage), the off-state effective capacitance seen to ground can be reduced by an order of magnitude relative to the MOSFET's Cₒₛₛ, thereby shortening the interval during which the capacitors C1, C2, C3, C4 remain effectively connected.

To determine which of the capacitors C1, C2, C3, C4 to connect to the circuit 300 to minimize a difference between the drive frequency and the resonant frequency, a current drawn by the circuit 300 as a function of the added capacitance can be monitored, and the capacitance that maximizes the monitored current can be determined, because the current is maximized when the resonant frequency is equal to the resonant frequency. In some cases, when the one or more capacitors 314 includes N capacitors having values of C₀*2ⁿ, for n=0 to N-1, each of the 2^{N} possible values of the total capacitance can be applied to the circuit, and the value that maximizes the current drawn by the circuit can be selected to apply to the circuit.

In some implementations, a more efficient algorithm can be used to determine the value of the capacitance that maximizes the current drawn by the circuit 300. For example, in some implementations, a golden section algorithm can be used. The golden section algorithm is an efficient technique to progressively narrow the interval of the capacitance values over which the current drawn by the circuit is evaluated to find the capacitance value that maximizes the current as a function of the capacitance. When the function has only one maximum over the range of capacitance values (as with equation (4)), the maximum is found within the interval defined by the two points adjacent to the point at which the greatest value has been determined. The golden search algorithm operates by successively narrowing the range of capacitance values on the specified interval in which the value of the function is evaluated and derives its name from the fact that the algorithm evaluates the function values for four points whose three interval widths are p: 1:0, where q is the golden ratio of approximately 1.61803398. These ratios are maintained for each iteration of the algorithm, and the algorithm can be repeated until the interval in which the maximum occurs is only one step wide (e.g., equal to C₀) at which point the capacitance value of the two values that define that interval and that causes a higher current to be drawn can be selected as the capacitance value to use for the capacitance provided by the one or more second capacitors 314.

The golden ratio algorithm is a limit of a Fibonacci search algorithm, which also can be used to determine the capacitance that maximizes the current drawn by the circuit 300 and therefore sets the resonant frequency of the circuit to the drive frequency. In the Fibonacci search algorithm, the length of the interval the spans the number of discrete capacitance values that are possible using with the circuit 400 can be set a Fibonacci number, FN, that is greater than the total number of discrete capacitance values, and the three initial capacitance values at which current is measured can be the minimum capacitance value, the maximum capacitance value, and the capacitance value that corresponds to the point that divides the interval between the minimum and maximum values into a first interval that has a length of the next lower Fibonacci number, F_{(N-1)} and a second interval that has a length of the second next lower Fibonacci number, F_{(N-2)}. Then, a point can be defined in the first interval that divides the first interval into sub intervals having lengths of the next two lower Fibonacci numbers. The algorithm can be iterated until a maximum of the current is determined, and then the capacitance at which the current maximum occurs can be selected as the capacitance to be provided by the one or more second capacitors 314.

In some implementations, the algorithm to determine the capacitance to add to the circuit can be performed once the external charger is placed in proximity to, and in alignment with, the implantable medical device and a current provided to the transmission coil has exceeded a threshold current. Thus, the resonant frequency of the energy transmission circuit of the external charger can be tuned once the transmission coil is already providing energy to the implantable medical device and can be used to fine-tune the resonant frequency of the energy transmission circuit to the drive frequency of the external charger when the charger is providing energy to the implantable medical device. The algorithm can be performed periodically as the external charger provides energy to the implantable medical device so as to update the resonant frequency of the energy transmission circuit as a mutual inductance between the external charger and the implantable medical device changes, for example, due to changes in relative location or alignment between the charger and the medical device, due to changes in the amount of power transmitted from the charger to the medical device, etc.

Referring again to FIG. 3, the circuit 300 can include one or more third capacitors 318 that can be rapidly switched into the circuit to adjust the capacitance of the circuit and thereby adjust the resonant frequency of the circuit. In an implementation, the one or more capacitors 318 can be connected to the circuit for a first integer number of periods of the drive frequency and disconnected from the circuit for a second integer number of periods of the drive frequency. The one or more capacitors 318 can be connected and disconnected to the circuit 300 during the dead time of the H-bridge (i.e., when zero voltage is provided to the transmission coil 302) to reduce losses. The one or more capacitors 318 can be connected to, and disconnected from, the circuit 300 with a frequency that is less than the drive frequency, and the ratio between the first number of periods and the sum of the first and second number of periods can define a duty cycle (DC) with which the one or more capacitors 318 are connected to the circuit 300. The duty cycle can be controlled (e.g., through a pulse width modulation (PWM) process) between 0% and 100% to control the time-averaged capacitance that is added to the circuit by dynamically switching the one or more capacitors 318 into connection with the circuit, and the time-averaged capacitance can control the resonant frequency of the circuit to be equal to the drive frequency of the circuit.

In addition to using PWM techniques to vary the time-averaged capacitance provided to the circuit 300 by the one or more capacitors 318, pulse frequency modulation (PFM) techniques also can be used. In PFM, the one or more capacitors 318 can be connected to the circuit 300 for a time period equal to an integer number of periods of the drive frequency and the frequency with which the one or more capacitors are connected to the circuit can be varied to control the duty cycle with which the one or more capacitors 318 are connected to the circuit 300. Thus, the PFM techniques can be used to control the time-averaged capacitance added to the circuit and thereby to control the resonant frequency of the circuit to be equal to the drive frequency of the circuit.

In some implementations, static adjustments of the capacitance of the circuit 300 can be provided through the relatively slow switching of the one or more capacitors 314 into the circuit and dynamic adjustments of the capacitance of the circuit 300 can be provided through the relatively fast switching of the one or more capacitors 318 into the circuit. In some implementations, static adjustments of the capacitance of the circuit 300 can be provided through the relatively slow switching of the one or more capacitors 314 into the circuit without providing dynamic adjustments of the capacitance. In some implementations, dynamic adjustments of the capacitance of the circuit 300 can be provided through the relatively fast switching of the one or more capacitors 318 into the circuit without providing static adjustments of the capacitance of the circuit.

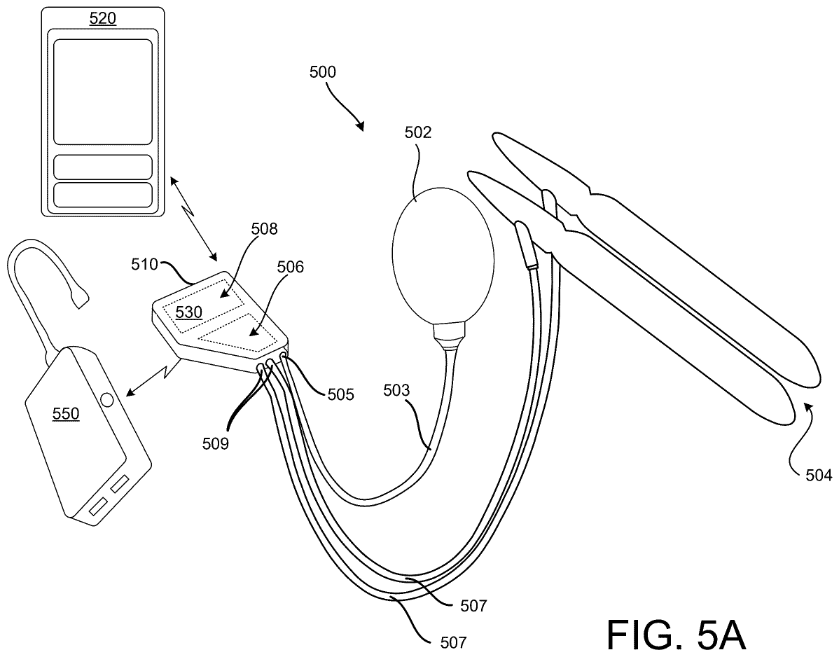

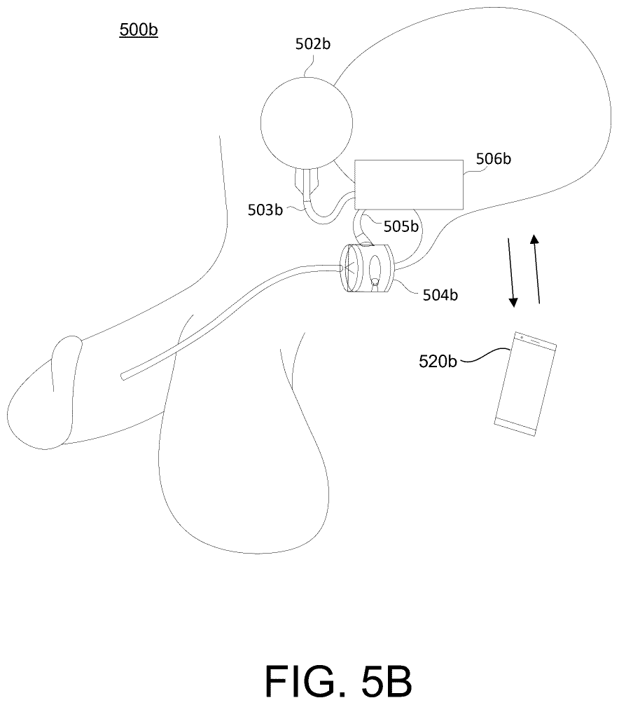

FIG. 5A illustrates an example implantable fluid-operated inflatable device 500 in the form of an example inflatable penile prosthesis. The example inflatable device 500 may be an example of the medical device 100 of FIGS. 1A to 1F and may include any of the details discussed with reference to those figures. In some examples, the example implantable fluid-operated inflatable device 500 may be charged using the power transmission device 150 of FIGS. 1A to 1F. In some examples, the power receiver 130 (including the receiver member 132), the electronic circuitry 134, battery 136, and/or the sensor(s) 141 are included in the fluid control system 506.

The example inflatable device 500 includes a fluid control system 506 including fluidics components such as pumps, valves, sensing devices and the like positioned in fluid passageways. In some implementations, the fluid control system includes components such as, for example, one or more fluid control devices, one or more pressure sensors, and other such components. In some implementations, the example inflatable device 500 includes an electronic control system 508 configured to provide for the transfer of fluid between a reservoir 502 and an inflatable member 504 via the fluidics components. In the example shown in FIG. 5A, the inflatable member 504 is in the form of a pair of inflatable cylinders. In the example shown in FIG. 5A, fluidics components of the fluid control system 506, and electronic components of the electronic control system 508 are received in a housing 510. In some implementations, fluidics components of the fluid control system 506, and electronic components of the electronic control system 508 received in the housing 510 together define an electronically controlled fluid manifold 530 that provides for the electronic control of the flow of fluid between the reservoir 502 and the inflatable member 504.

In the example shown in FIG. 5A, a first conduit 503 connects a first fluid port 505 of the electronically controlled fluid manifold 530 (the fluid control system 506/electronic control system 508 received in the housing 510) with the reservoir 502. One or more second conduits 507 connect one or more second fluid ports 509 of the electronically controlled fluid manifold 530 (the fluid control system 506/electronic control system 508 received in the housing 510) with the inflatable member 504 in the form of the inflatable cylinders. In some examples, the electronic control system 508 can communicate with an external controller 520, via respective communication modules. For example, an application stored in a memory and executed by a processor of the external controller 520 may allow the user and/or a physician to operate, view, monitor and alter operation of the inflatable device 500. In some examples, components of the electronic control system 508 and/or the fluid control system 506 can be charged and/or recharged by a power transmission device (e.g., the power transmission device 150 of FIGS. 1A to 1F) of the external controller 520, and/or by a power transmission device 550 (e.g., the power transmission device 150 of FIGS. 1A to 1F), that is separate from the external controller 520.

The example implantable fluid-operated inflatable device 500 shown in FIG. 5A includes an electronic control system 508 to provide for control of the operation of the respective inflatable members 504 in the form of cylinders, and the monitoring and control of pressure and/or fluid flow through inflatable members 504.

FIG. 5B illustrates a urinary control device 500b having an electronic pump assembly 506b according to an aspect. In some examples, the urinary control device 500b is an artificial urinary sphincter device. The electronic pump assembly 506b may include any of the features of the electronic pump assembly discussed herein, including fluid control system 506 of FIG. 5A. The example urinary control device 500b may be an example of the medical device 100 of FIGS. 1A to 1F and may include any of the details discussed with reference to those figures. The urinary control device 500b includes a pump assembly 506b, a fluid reservoir 502b, and a cuff 504b (e.g., an inflatable cuff). In some examples, the urinary control device 500b may be charged using the power transmission device 150 of FIGS. 1A to 1F. In some examples, the power receiver 130 (including the receiver member 132), the electronic circuitry 134, battery 136, and/or the sensor(s) 141 are included in the pump assembly 506b.

The fluid reservoir 502b may be a pressure-regulating inflation balloon or element. The fluid reservoir 502b is in operative fluid communication with the cuff 504b via one or more tube members 503b, 505b. The fluid reservoir 502b is constructed of polymer material that is capable of elastic deformation to reduce fluid volume within the fluid reservoir 502b and push fluid out of the fluid reservoir 502b and into the cuff 504b. However, the material of the fluid reservoir 502b can be biased or include a shape memory construct adapted to generally maintain the fluid reservoir 502b in its expanded state with a relatively constant fluid volume and pressure. In some examples, this constant level of pressure exerted from the fluid reservoir 502b to the cuff 504b will keep the cuff 504b at a desired inflated state when open fluid communication is provided between the fluid reservoir 502b and the cuff 504b. In some examples, the fluid reservoir 502b is implanted into the abdominal space.

A user may use an external device 520b to control the urinary control device 500b. In some examples, the user may use the external device 520b to inflate or deflate the cuff 504b. For example, in response to the user activating an inflation cycle using the external device 520b, the external device 520b may transmit a wireless signal to the electronic pump assembly 506b to initiate the inflation cycle to transfer fluid from the fluid reservoir 502b to the cuff 504b (e.g., by opening an active valve where the pressure in the fluid reservoir 502b causes the fluid to move through the active valve to the cuff 504b). In some examples, in response to the user activating a deflation cycle using the external device 520b, the external device 520b may transmit a wireless signal to the electronic pump assembly 506b to initiate the deflation cycle to transfer fluid from the cuff 504b to the fluid reservoir 502b.

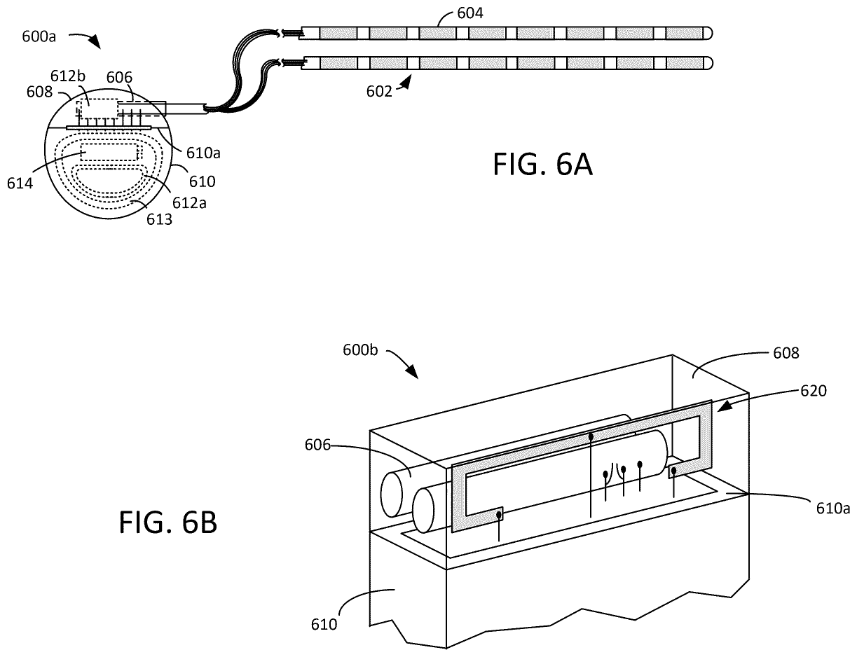

FIGS. 6A and 6B show different examples of implantable neurostimulators 600a and 600b that can be charged or powered by the disclosed power transmission device 150. Examples of these implantable neurostimulators are disclosed in U.S. patent application Ser. No. 18/658,543, filed May 8, 2024, which is incorporated herein by reference in its entirety. Implantable neurostimulators 600a and 600b may be, for example, used to provide Spinal Cord Stimulation (SCS) or Deep Brain Stimulation (DBS).

Implantable neurostimulator 600a comprise a biocompatible device case 610 formed of a conductive material such as titanium, for example. The case 610 typically holds the circuitry and power source (e.g., a battery 614) necessary for the neurostimulator to function, although the neurostimulator 600a also can be powered by the disclosed power transmission device 150 continually and therefore may lack a battery. The neurostimulator 600a is coupled to electrodes 604 via one or more electrode leads 602, such that the electrodes form an electrode array. The leads 602 connect to neurostimulator 600a using lead connectors 606, which are fixed in a non-conductive header material 608 such as a non-conductive epoxy, for example. Contacts at the proximal ends of the leads 602 connect to contacts in the lead connectors 606, which are in turn connected to feedthrough wires spanning between the header 608 and the case 610, and ultimately to circuitry (e.g., a circuit board) inside the case 610. The neurostimulator 600a can be programmed to provide electrical stimulation to nervous tissue via any one or more the electrodes 604.

The neurostimulator 600a as shown in FIG. 6A may include one or more telemetry antennas 612a and/or 612b used to wirelessly transmit/receive data to/from an external communication system, such as a patient external controller or a clinician programmer. In some implementations, the telemetry antenna 612a is in the case 610, and may comprise a coil, although this antenna 612a could also be located in the header 608. When configured as a coil, the antenna 612a can communicate data with the external communication system via magnetic induction. The telemetry antenna 612b is in the header 608 but could also be located in the case. The telemetry antenna 612b may comprise a wire, slot, or patch antenna to receive and transmit data using far-field electromagnetic fields (e.g., at 2.4 GHz). Antennas 612a and 612b can communicate using a protocol, such as Frequency Shift Keying or Bluetooth, for example.

The neurostimulator 600a may also include a receiver member 613 for wirelessly receiving the magnetic field (power) from a power transmission device such as 150. In this example, the receiver member 613 can include a receiver coil that receives power from the power transmission device 150 by magnetic induction. The receiver member 613 is shown within the case 610 but may also be located in the header 608. A surface 610a (or a surface portion) may exist between the case 610 and the header 608. In some examples, the receiver member 613 may be an example of the receiver member 132 of FIGS. 1A to 1F and may include any of the details discussed with reference to those figures. The neurostimulator 600a can include rectification circuitry for converting current induced in the receiver member 613 by the received power to a DC current that powers the neurostimulator directly and/or charges its battery 614. When configured as a coil, the receiver member 613 can receive power from the power transmission device 150 via a magnetic field having a relatively low frequency, such as less than 150 kHz, less than 60 kHz, or equal to or less than 80 KHz.

Neurostimulator 600b as shown in FIG. 6B discloses a different antenna structure 620 that can act as both a receiver and transmitter of data, and as a receiver member for receiving power from a power transmission device such as 150. As explained in the above-incorporated '543 Application, this antenna structure 620 preferably comprises at least one planar sheet of metal formed (e.g., by stamping or milling) into the requisite shape. The antenna structure 620 in this example is provided in the non-conductive header 608. As explained in the '543 Application, the antenna structure 620 can both communicate data via far-field electromagnetic fields (e.g., at 2.4 GHz) with an external communication system, and can receive power from a power transmission device such as 150 via magnetic induction. The power received from the power transmission device 150 can be provided through and alternating current signal having a frequency of, for example, greater 6 MHz, or equal to 6.78 MHz, or equal to 13.56 MHz in the Industrial, Scientific, and Medical (ISM) radio band.

While certain features of the described implementations have been illustrated as described herein, many modifications, substitutions, changes and equivalents will now occur to those skilled in the art. It is, therefore, to be understood that the will and in and in appended claims are intended to cover all such modifications and changes as fall within the scope of the embodiments.

What is claimed is:

1. An external charger configured for charging a battery of an implantable medical device, the external charger comprising:
a transmission coil configured for generating a time-varying magnetic field with a driving frequency;
an electrical circuit for providing an alternating current (AC) waveform to the transmission coil, wherein providing the AC waveform to the transmission coil causes the transmission coil to generate the time-varying magnetic field, the electrical circuit having a resonant frequency;
one or more capacitors;
one or more switches configured to switch the one or more capacitors into the electrical circuit; and
a controller configured for controlling the one or more switches to switch the one or more capacitors into the circuit to maintain the resonant frequency of the circuit equal to the driving frequency.

2. The external charger of claim 1,
wherein the one or more capacitors includes at least a first capacitor having a capacitance, C, a second capacitor having a capacitance, 2C, and a third capacitor having a capacitance, 4C, the first, second, and third capacitors being arranged in parallel with each other,
wherein the one or more switches includes a first switch connected in series with the first capacitor, a second switch connected in series with the second capacitor and a third switch connected in series with the third capacitor,
wherein the controller is configured to control the switches to add a capacitance of C, 2C, 3C, 4C, 5C, 6C or 7C to the electrical circuit.

3. The external charger of claim 2, further comprising a current sensing circuit configured to sense a current provided to the transmission coil,
wherein the controller is configured to determine the capacitance to add to the electrical circuit based on the current sensed by the sensing circuit.

4. The external charger of claim 3, wherein the controller is configured to:
control the switches to add different discrete capacitances to the electrical circuit,
receive signals from the current sensing circuit indicating a current provided to the transmission coil when the different discrete capacitances are added to the circuit,
execute an algorithm, based on the received signals, to determine a capacitance that maximizes the current provided to the transmission coil, and
control the switches to add the capacitance that maximizes the current provided to the transmission coil to maintain the resonant frequency equal to the driving frequency.

5. The external charger of claim 3, when the charger is configured to:
receive a signal from the current sensing circuit indicating a current provided to the transmission coil is greater than a threshold current, and
when the current provided to the transmission coil is greater than the threshold current;
control the switches to add different discrete capacitances to the electrical circuit,
receive signals from the current sensing circuit indicating a current provided to the transmission coil when the different discrete capacitances are added to the circuit,
execute an algorithm, based on the received signals, to determine a capacitance that maximizes the current provided to the transmission coil, and
control the switches to add the capacitance that maximizes the current provided to the transmission coil to maintain the resonant frequency equal to the driving frequency.

6. The external charger of claim 5, wherein the algorithm includes a Fibonacci search algorithm.

7. The external charger of claim 4,
wherein the one or more capacitors includes at least a fourth capacitor having a capacitance, C₀,
wherein the one or more switches includes a fourth switch connected in series with the fourth capacitor,
wherein the controller is configured to control the fourth switch to periodically switch the fourth switch between a closed state and an open state, the switching occurring during dead time periods of the electrical circuit that provides the alternating current waveform to the transmission coil, the switching occurring with a duty cycle (DC) and causing a time-averaged capacitance equal to DC* C₀ to be added to the electrical circuit.

8. The external charger of claim 7, wherein the duty cycle is determined by controlling a length of time with which the fourth switch is in a closed state during the periodic switching of the fourth switch.

9. The external charger of claim 7, wherein the duty cycle is determined by controlling a frequency with which the fourth switch is switched between the open state and the closed state during the periodic switching of the fourth switch.

10. The external charger of claim 1, wherein the implantable medical device includes an inflatable penile prosthesis, a urinary control device, or a neurostimulation medical implant.

11. A method of controlling an external charger configured to charge a battery of an implantable medical device, the method comprising:
providing an alternating current (AC) waveform having a driving frequency from an electrical circuit to a transmission coil to cause the transmission coil to generate a time-varying magnetic field, the electrical circuit having a resonant frequency; and
controlling one or more switches to switch one or more capacitors into the electrical circuit to maintain the resonant frequency of the circuit equal to the driving frequency while the AC waveform is provided from the electrical circuit to the transmission coil.

12. The method of claim 11,
wherein the one or more capacitors includes at least a first capacitor having a capacitance, C, a second capacitor having a capacitance, 2C, and a third capacitor having a capacitance, 4C, the first, second, and third capacitors being arranged in parallel with each other,
wherein the one or more switches includes a first switch connected in series with the first capacitor, a second switch connected in series with the second capacitor and a third switch connected in series with the third capacitor, and the method further comprising:
controlling the switches to add a capacitance of C, 2C, 3C, 4C, 5C, 6C or 7C to the electrical circuit.

13. The method of claim 12, further comprising:
sensing a current provided to the transmission coil; and
controlling the switches to add the capacitance of C, 2C, 3C, 4C, 5C, 6C or 7C to the electrical circuit based on the sensed current.

14. The method of claim 13, further comprising:
controlling the one or more switches to add different discrete capacitances to the electrical circuit;
sensing the current provided to the transmission coil when the different discrete capacitances are added to the circuit;
executing an algorithm, based on the sensed current, to determine a capacitance that maximizes the current provided to the transmission coil; and
controlling the switches to add the capacitance that maximizes the current provided to the transmission coil to maintain the resonant frequency equal to the driving frequency.

15. The method of claim 13, further comprising:
sensing that a current provided to the transmission coil is greater than a threshold current, and
when the current provided to the transmission coil is greater than the threshold current;
controlling the switches to add different discrete capacitances to the electrical circuit;
sensing the current provided to the transmission coil when the different discrete capacitances are added to the circuit;
executing an algorithm, based on the sensed current, to determine a capacitance that maximizes the current provided to the transmission coil; and
controlling the switches to add the capacitance that maximizes the current provided to the transmission coil to maintain the resonant frequency equal to the driving frequency.

16. The method of claim 15, wherein the algorithm includes a Fibonacci search algorithm.

17. The method of claim 14,
wherein the one or more capacitors includes at least a fourth capacitor having a capacitance, C₀,
wherein the one or more switches includes a fourth switch connected in series with the fourth capacitor, and the method further comprising:
controlling the fourth switch to periodically switch the fourth switch between a closed state and an open state, the switching occurring during dead time periods of the electrical circuit that provides the alternating current waveform to the transmission coil, the switching occurring with a duty cycle (DC) and causing a time-averaged capacitance equal to DC* C₀ to be added to the electrical circuit.

18. The method of claim 17, wherein the duty cycle is determined by controlling a length of time with which the fourth switch is in a closed state during the periodic switching of the fourth switch.

19. The method of claim 17, wherein the duty cycle is determined by controlling a frequency with which the fourth switch is switched between the open state and the closed state during the periodic switching of the fourth switch.

20. The method of claim 11, where the implantable medical device includes an inflatable penile prosthesis, a urinary control device, or a neurostimulation medical implant.
